(19) United States
(12) Patent Application Publication
(10) Pub. No.: US 2025/0160350 A1
(43) Pub. Date: May 22, 2025

(54) BIOACTIVE MOLECULES IN YOGURT

(71) Applicant: Phytoquest Limited, Aberystwyth (GB)

(72) Inventors: Robert James Nash, Ystrad-Meurig (GB); Yana Penkova, Ystrad-Meurig (GB)

(73) Assignee: Phytoquest Limited, Aberystwyth (GB)

(21) Appl. No.: 18/921,749

(22) Filed: Oct. 21, 2024

Related U.S. Application Data
(63) Continuation of application No. PCT/GB2023/051076, filed on Apr. 24, 2023.

(30) Foreign Application Priority Data
Apr. 22, 2022 (GB) 2205846.5

(51) Int. Cl. A23C 9/123; A61K 31/4168; C12Q 1/34

(52) U.S. Cl. CPC A23C 9/1238; A61K 31/4168; C12Q 1/34

(57) ABSTRACT

Described are compositions comprising a cyclic guanidine for use in a method of neuroprotection or for treating or preventing a GBA deficiency disease. Also described are pharmaceutical compositions comprising a cyclic guanidine and a pharmaceutically acceptable excipient, along with related methods of treating or preventing a GBA deficiency disease. Also described are various processes for producing a yogurt, or for monitoring the quality of a yogurt, comprising testing the sample for GCase activity, the presence of glycosidase modulatory activity, the presence or absence of a cyclic guanidine and/or to measure the amount of a cyclic guanidine in the sample, and/or for the presence of glycosidase modulatory activity, for example for the inhibition or promotion of one or more glycosidases.

### FIELD OF THE INVENTION

The present invention relates to compositions comprising a bioactive cyclic guanidine found in yogurt, processes for the production of pharmaceutical compositions comprising the bioactive cyclic guanidine and to processes for producing foods (including in particular yogurts) which involve testing for the presence and/or activity of the bioactive cyclic guanidine.

### BACKGROUND OF THE INVENTION

Yogurt is a food produced by the bacterial fermentation of milk. The bacterial fermentation is initiated by the use of yogurt starter cultures. Starter cultures are a blend of different bacteria, typically comprising Lactobacillus delbrueckii subsp. bulgaricus and Streptococcus thermophilus.

Yogurts have long been considered to have beneficial effects on health, largely on the basis of anecdotal evidence. More recently, data has emerged demonstrating positive probiotic effects on the constitution and/or activity of the gut microbiome, exerted at least in part via immunomodulatory effects. Thus, yogurt is considered to be a functional food, offering health benefits in addition to its basic nutritional qualities.

Accordingly, there is presently much interest in the viability of bacteria in the yogurt itself, as well as their persistence and fate in the gastrointestinal tract following consumption and their effects on the gut microbiome.

However, there is very little information on the possible contribution of bioactive molecules (particularly small molecules produced during bacterial fermentation) to the positive physiological effects.

Moreover, processes for producing yogurt typically focus on the selection of the milk source, milk pre-processing protocols, the nature of the starter culture and fermentation parameters, with quality control carried out by measurements of viscosity (and other textural qualities) during milk preparation and processing, during fermentation and prior to packaging. Quality control may also extend to assessments of organoleptic qualities (especially taste, texture and mouthfeel).

There is therefore a need for a greater understanding of the role of bioactive small molecules in the health benefits of yogurt, as well as improved methods for monitoring the biochemical profile of yogurt so that its functional potential in vivo after consumption can be monitored and the health promoting effects of yogurt as a functional food more reliably delivered.

### SUMMARY OF THE INVENTION

The present invention is based, at least in part, on the surprising discovery of an iminosugar-like cyclic guanidine compound in yogurt that selectively promotes glucocerebrosidase (GCase) activity. Without wishing to be bound by any theory, it is believed that the GCase promoting activity may involve the stabilization of an active conformation via binding to non-catalytic sites (and chaperone activity in vivo).

GCase activity plays an important role in neuroprotection and a reduction, or loss of, GCase activity plays an important role in many diseases (especially neurodegenerative diseases). For example, GCase deficiency is a key mechanism in Lewy body disease (LBD), Parkinson's disease (PD), dementia with Lewy bodies (DLB), Alzheimer's disease (AD) and Gaucher's disease (GD).

Thus, for the first time, a small molecule has been identified as an important bioactive principle in yogurt. This is of fundamental importance, since it provides a new class of drugs for the treatment of neurodegenerative diseases and permits the functional properties of yogurt to be optimized via appropriate quality control and process design.

During work on the characterization of glycosidase modulatory profile of yogurt and yogurt fractions, it was also discovered that yogurts contain other bioactive factors which modulate glycosidases, with some glycosidase enzymes inhibited and others promoted. This is also of great commercial importance, since it permits further optimization of the functional properties of yogurt by testing for glycosidase modulation.

Thus, according to a first aspect of the present invention there is provided a composition comprising a cyclic guanidine for use in a method of neuroprotection or for treating or preventing a GBA deficiency disease.

Some of the cyclic compounds of the invention are new chemical entities. Accordingly, according to a second aspect of the invention there is provided a composition comprising a cyclic guanidine glucocerebrosidase activator.

According to a third aspect of the invention, there is provided a pharmaceutical composition comprising a cyclic guanidine of the invention and a pharmaceutically acceptable excipient.

In a fourth aspect of the invention, there is provided a method of neuroprotection or for treating or preventing a GBA deficiency disease comprising administering an effective amount of the composition or cyclic guanidine of the invention to a subject, for example to a human subject.

In a fifth aspect of the invention, there is provided the use of the composition or cyclic guanidine of the invention for the manufacture of a medicament for neuroprotection or for treating or preventing a GBA deficiency disease.

In a sixth aspect of the invention, there is provided a process for producing a yogurt, or for monitoring the quality of a yogurt, comprising the steps of: (a) providing a yogurt sample; and (b) testing the sample for GCase activity or for the presence of glycosidase modulatory activity, for example for the inhibition or promotion of one or more glycosidases.

In a seventh aspect of the invention, there is provided a process for producing a yogurt, or for monitoring the quality of a yogurt, comprising the steps of: (a) providing a yogurt sample; and (b) testing the sample for the presence or absence of a cyclic guanidine, or to measure the amount of a cyclic guanidine in the sample.

In an eighth aspect of the invention, there is provided a process for producing a yogurt, or for monitoring the quality of a yogurt, comprising the steps of: (a) providing a yogurt sample; (b) testing the sample for GCase activity; and (c) testing the sample for the presence or absence of a cyclic guanidine, or to measure the amount of a cyclic guanidine in the sample.

In a ninth aspect of the invention, there is provided a process for producing a yogurt, comprising the steps of: (a) providing a milk product; (b) fermenting the milk product, for example with bacteria comprising Lactobacillus delbrueckii subsp. bulgaricus, to yield a yogurt; (c) providing a sample of the yogurt of step (b); and (d) testing the sample: (i) for GCase activity; and/or (ii) for the presence or absence of a cyclic guanidine; and/or (iii) to measure the amount of a cyclic guanidine in the sample; and/or (iv) for the presence of glycosidase modulatory activity, for example for the inhibition or promotion of one or more glycosidases.

In a tenth aspect of the invention, there is provided a process for producing a yogurt, or for monitoring the quality of a yogurt, comprising the steps of: (a) providing a yogurt sample; and (b) testing the sample for the presence of glycosidase modulatory activity, for example for the inhibition or promotion of one or more glycosidases.

Other aspects and preferred embodiments of the invention are defined and described in the claims set out below.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### I. Definitions

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers. Thus, as used herein the term “comprising” is inclusive or open-ended and does not exclude additional, unrecited integers or method/process steps.

The phrase “consisting essentially of” is used herein to require the specified integer(s) or steps as well as those which do not materially affect the character or function of the claimed invention.

As used herein, the term “consisting” is used to indicate the presence of the recited integer (e.g., a feature, element, characteristic, property, method/process step or limitation) or group of integers (e.g., features, element, characteristics, properties, method/process steps or limitations) alone.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g., the administration of an agent to a subject) which cures, ameliorates or lessens the symptoms of a disease or removes (or lessens the impact of) its cause(s) (for example, pathological states). In this case, the term is used synonymously with the term “therapy”.

Additionally, the terms “treatment” or “treating” refers to an intervention (e.g., the administration of an agent to a subject) which prevents or delays the onset or progression of a disease or reduces (or eradicates) its incidence within a treated population. In this case, the term treatment is used synonymously with the term “prophylaxis”.

The term “subject” (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals and pet animals. In preferred embodiments, the subject is a human.

As used herein, an effective amount of a compound or composition defines an amount that can be administered to a subject without excessive toxicity, irritation, allergic response, or other problem or complication, commensurate with a reasonable benefit/risk ratio, but one that is sufficient to provide the desired effect, e.g., the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

The term isolated as applied to the compounds of the invention is used herein to indicate that the compound exists in a physical milieu distinct from that in which it occurs in nature.

For example, the isolated compound may be substantially isolated (for example enriched or purified) with respect to the complex cellular milieu in which it naturally occurs. The isolated compound may therefore take the form of an enriched fraction or extract of any of the sources (e.g., yogurt sources) described herein.

When the isolated material is enriched or purified, the absolute level of enrichment or purity is not critical and those skilled in the art can readily determine appropriate levels according to the use to which the material is to be put. Preferred are purity levels of at least 0.1% w/w, 0.2% w/w, 0.3% w/w, 0.4% w/w, 0.5% w/w, 0.6% w/w, 0.7% w/w, 0.8% w/w, 0.9% w/w, 1.0% w/w, 1.1% w/w, 1.2% w/w, 1.3% w/w, 1.4% w/w, 1.5% w/w, 1.6% w/w, 1.7% w/w, 1.8% w/w, 1.9% w/w or 2.0% w/w.

Particularly preferred are purity levels of at least 0.5-2.0% w/w, for example at least 0.8-1.5% w/w, for example at least about 1.0% w/w. Levels of 5-10% w/w may be readily obtained in cases where the material is isolated from natural sources, if necessary, by employing suitable enrichment techniques, such as ion exchange chromatography.

In some circumstances, the isolated compound forms part of a composition (for example a more or less crude extract containing many other substances) or buffer system, which may for example contain other components. In other circumstances, the isolated compound may be purified to essential homogeneity, for example as determined spectrophotometrically, by NMR or by chromatography (for example GC-MS of the trimethylsilyl-derivatives).

The terms derivative and pharmaceutically acceptable derivative as applied to the compounds of the invention define compounds which are obtained (or obtainable) by chemical derivatization of the parent compound of the invention. The pharmaceutically acceptable derivatives are therefore suitable for administration to or use in contact with the tissues of humans without undue toxicity, irritation or allergic response (i.e., commensurate with a reasonable benefit/risk ratio). Preferred derivatives are those obtained (or obtainable) by alkylation, esterification or acylation of the parent compounds.

The pharmaceutically acceptable derivatives of the invention may retain some or all of the biological activities described herein. In some cases, the biological activity (e.g., GCase chaperone activity) is increased by derivatization. The derivatives may act as pro-drugs, and one or more of the biological activities described herein (e.g., pharmacoperones activity) may arise only after in vivo processing. Derivatization may also augment other biological activities of the compound, for example bioavailability and/or glycosidase promoting/inhibitory activity and/or its glycosidase modulatory profile. For example, derivatization may increase glycosidase inhibitory potency and/or specificity and/or CNS penetration (e.g., penetration of the blood-brain barrier).

The term pharmaceutically acceptable salt as applied to the cyclic guanidines of the invention defines any non-toxic organic or inorganic acid addition salt of the free base which are suitable for use in contact with the tissues of humans and lower animals without undue toxicity, irritation, allergic response and which are commensurate with a reasonable benefit/risk ratio. Suitable pharmaceutically acceptable salts are well known in the art.

Examples are the salts with inorganic acids (for example hydrochloric, hydrobromic, sulphuric and phosphoric acids), organic carboxylic acids (for example acetic, propionic, glycolic, lactic, pyruvic, malonic, succinic, fumaric, malic, tartaric, citric, ascorbic, maleic, hydroxymaleic, dihydroxymaleic, benzoic, phenylacetic, 4-aminobenzoic, 4-hydroxybenzoic, anthranilic, cinnamic, salicylic, 2-phenoxybenzoic, 2-acetoxybenzoic and mandelic acid) and organic sulfonic acids (for example methanesulfonic acid and p-toluenesulfonic acid).

These salts and the free base compounds can exist in either a hydrated or a substantially anhydrous form. Crystalline forms, including all polymorphic forms, of the compounds of the invention are also contemplated and in general the acid addition salts of the compounds are crystalline materials which are soluble in water and various hydrophilic organic solvents and which in comparison to their free base forms, demonstrate higher melting points and an increased solubility.

The term pharmaceutically acceptable metabolite as applied to the compounds of the invention defines a pharmacologically active product produced through metabolism in the body of the specified compound or salt thereof.

The term pharmaceutically acceptable prodrug as applied to the compounds of the invention defines any pharmaceutically acceptable compound that may be converted under physiological conditions or by solvolysis to the specified compound, to a pharmaceutically acceptable salt of such compound or to a compound that shares at least some of the activity of the specified compound. Prodrugs and active metabolites of the compounds of the invention may be identified using routine techniques known in the art (see for example, Bertolini et al., J. Med. Chem., 1997, 40, 2011-2016).

The term bioisostere (or simply isostere) is a term of art used to define drug analogues in which one or more atoms (or groups of atoms) have been substituted with replacement atoms (or groups of atoms) having similar steric and/or electronic features to those atoms which they replace. The substitution of a hydrogen atom or a hydroxyl group with a fluorine atom is a commonly employed bioisosteric replacement. Sila-substitution (C/Si-exchange) is a relatively recent technique for producing isosteres. This approach involves the replacement of one or more specific carbon atoms in a compound with silicon (for a review, see Tacke and Zilch (1986) Endeavour, New Series 10: 191-197). The sila-substituted isosteres (silicon isosteres) may exhibit improved pharmacological properties, and may for example be better tolerated, have a longer half-life or exhibit increased potency (see for example Englebienne (2005) Med. Chem., 1(3): 215-226). Similarly, replacement of an atom by one of its isotopes, for example hydrogen by deuterium, may also lead to improved pharmacological properties, for example leading to longer half-life (see for example Kushner et al (1999) Can J Physiol Pharmacol. 77(2):79-88). In its broadest aspect, the present invention contemplates all bioisosteres (and specifically, all silicon bioisosteres) of the compounds of the invention.

As used herein the term “promoting”, “elevating” or “enhancing” or variations thereof such as “promotion”, “elevation” or “elevatory” or “elevated” or “enhancement” or “enhancer” or “enhanced” in relation to GCase activity is defined as an increase by any value greater than 10%, for example greater than 10%, 20%, 50%, 60%, 70%, 80%, 90%, 100%, 150%, 200% or 500%.

As used herein the term “inhibits”, or variations thereof such as “inhibition” or “inhibiting” or “inhibitory” are to be defined as a decrease by any value greater than 10%, for example greater than 25%, 50%, 75% or 90%. It is to be understood that inhibiting does not require full inhibition.

The term bioactive principle is used herein to define a biomolecule which is necessary or sufficient for certain physiological effects of the natural product (typically, a yogurt) in which it is comprised. In the case of the present invention, the bioactive principle(s) comprise one or more of the cyclic guanidines of the invention.

In the present specification the term “alkyl” defines a straight or branched saturated hydrocarbon chain. The term “C1-C6 alkyl” refers to a straight or branched saturated hydrocarbon chain having one to six carbon atoms. Examples include methyl, ethyl, n-propyl, isopropyl, t-butyl, n-hexyl. The alkyl groups of the invention may be optionally substituted, e.g., by one or more halogen atoms.

In the present specification the term “alkenyl” defines a straight or branched hydrocarbon chain containing at least one carbon-carbon double bond. The term “C1-C6 alkenyl” refers to a straight or branched unsaturated hydrocarbon chain having one to six carbon atoms.

The alkenyl groups of the invention may be optionally substituted, e.g., by one or more halogen atoms.

In the present specification the term “alkynyl” defines a straight or branched hydrocarbon chain containing at least one carbon-carbon triple bond. The term “C1-C6 alkynyl” refers to a straight or branched unsaturated hydrocarbon chain having one to six carbon atoms. Examples include ethynyl, 2-propynyl, and 3-hexynyl. The alkynyl groups of the invention may be optionally substituted, e.g., by one or more halogen atoms.

### II. Cyclic Guanidines of the Invention

### Structural Aspects

As used herein, the term cyclic guanidine defines a compound which contains the following structural motif:

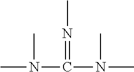

Thus, the cyclic guanidines of the invention may have the general formula I:

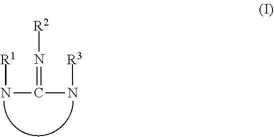

or a pharmaceutically acceptable salt, derivative, solvate, isomer, tautomer, N-oxide, ester, prodrug, isotope or protected form thereof, wherein R1, R2 and R3 are each independently selected from: H, methyl and optionally substituted C1-6 alkyl, C1-6 alkenyl or C1-6 alkynyl and wherein the connecting semi-circular line indicates one or more linking atoms/groups.

Also preferred are cyclic guanidines of general formula II:

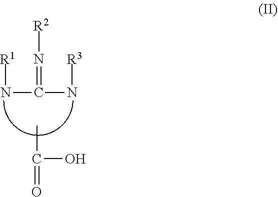

or a pharmaceutically acceptable salt, derivative, solvate, isomer, tautomer, N-oxide, ester, prodrug, isotope or protected form thereof, wherein R¹, R² and R³ are each independently selected from: H, methyl and optionally substituted C1-6 alkyl, C1-6 alkenyl or C1-6 alkynyl and wherein the connecting semi-circular line indicates one or more linking atoms/groups comprising a carboxyl group as shown.

Particularly preferred for use according to the invention is a cyclic guanidine of formula III:

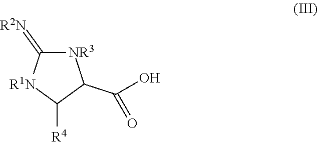

or a pharmaceutically acceptable salt, derivative, solvate, isomer, tautomer, N-oxide, ester, prodrug, isotope or protected form thereof, wherein R¹, R², R³ and R⁴ are each independently selected from: H and methyl.

Cyclic guanidines of formula III preferably have the following stereochemistry:

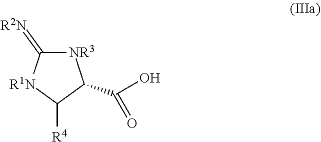

being otherwise as per formula III.

Examples of compounds of formula III for use according to the invention include:

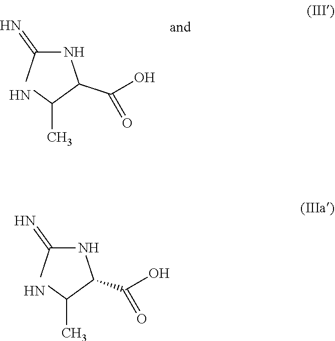

Also particularly preferred for use according to the invention is a cyclic guanidine of formula IV:

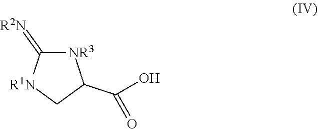

or a pharmaceutically acceptable salt, derivative, solvate, isomer, tautomer, N-oxide, ester, prodrug, isotope or protected form thereof, wherein R¹, R² and R³ are each independently selected from: H and methyl.

Cyclic guanidines of formula IV preferably have the following stereochemistry:

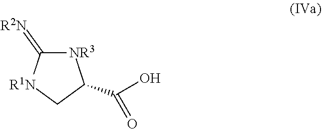

being otherwise as per formula IV.

More particularly preferred for use according to the invention is a cyclic guanidine of formula V:

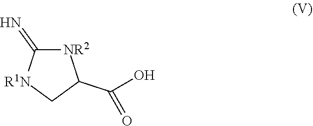

or a pharmaceutically acceptable salt, derivative, solvate, isomer, tautomer, N-oxide, ester, prodrug, isotope or protected form thereof, wherein R¹ and R² are each independently selected from: H and methyl.

Cyclic guanidines of formula V preferably have the following stereochemistry:

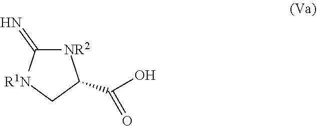

being otherwise as per formula V.

Also more particularly preferred for use according to the invention is a cyclic guanidine of formula VI:

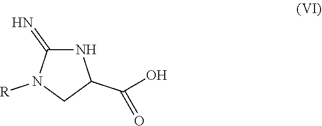

or a pharmaceutically acceptable salt, derivative, solvate, isomer, tautomer, N-oxide, ester, prodrug, isotope or protected form thereof, wherein R is H or methyl.

Cyclic guanidines of formula VI preferably have the following stereochemistry:

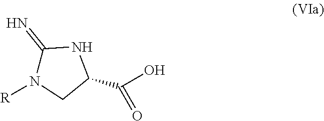

being otherwise as per formula VI.

Also more particularly preferred for use according to the invention is a cyclic guanidine of formula VII:

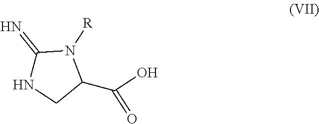

or a pharmaceutically acceptable salt, derivative, solvate, isomer, tautomer, N-oxide, ester, prodrug, isotope or protected form thereof, wherein R is H or methyl.

Cyclic guanidines of formula VII preferably have the following stereochemistry:

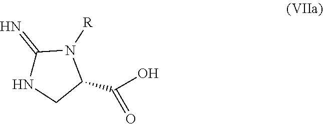

being otherwise as per formula VII.

Most particularly preferred is (S)-2-imino-3-methylimidazolidine-4-carboxylic acid of formula:

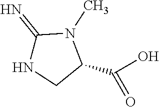

or a pharmaceutically acceptable salt, derivative, solvate, isomer, tautomer, N-oxide, ester, prodrug, isotope or protected form thereof, for example an isomer having the formula:

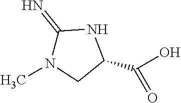

### Functional Aspects

The cyclic guanidines of the invention are preferably glucocerebrosidase (GCase) activators. Most preferred are cyclic guanidines which are activators of β-glucocerebrosidase, particularly human glucocerebrosidase (GCase; EC 3.2.1.45), also referred to as GBA or acid β glucocerebrosidase (encoded by the GBA1 gene). These may be referenced herein as GCase activating cyclic guanidines.

GCase activation can be measured in any of a wide range of suitable in vitro assays known to those skilled in the art. GCase activation may be recognized by an increase in activity versus a suitable control by any value greater than 10% (for example greater than 20%, 50%, 60%, 70%, 80%, 90%, 100%, 150%, 200% or 500%).

A preferred assay for use in characterizing GCase activation determines whether the cyclic guanidine increases the catalytic activity of human ß-glucocerebrosidase >50% in vitro in 0.1 M citric acid/0.2 M disodium hydrogen phosphate buffer at 27° C. and pH 5.9.

An exemplary assay is-uses human recombinant imiglucerase (obtained from Genzyme, Europe) and is conducted at 27° C. in 0.1 M citric acid/0.2 M disodium hydrogen phosphate buffer at pH 5.9. The incubation mixture consists of 10 μL of enzyme solution, 10 μL of aqueous solution of test material (e.g., yogurt, yogurt extract or isolated cyclic guanidine compound) at the required concentration and 50 μL of 5 mM para-nitrophenyl (PNP) B glucopyranoside (Sigma-Aldrich) made up in buffer at pH 5.9. The reactions are stopped by addition of 70 μL 0.4 M glycine (pH 10.4) during the exponential phase of the reaction, which is previously determined using uninhibited assays in which water replaces inhibitor. Final absorbances were read at 405 nm using a Versamax microplate reader (Molecular Devices). Assays are carried out in triplicate, and the values given are means of the three replicates per assay. A negative value indicates enzyme activation.

Without wishing to be bound by any theory, it is believed that the GCase promoting activity may arise via binding to a non-catalytic (e.g., allosteric) site, thereby stabilizing an active conformation (which may promote the adoption of a functional conformation during folding in vivo). Thus, the cyclic guanidines of the invention may act as pharmacoperones for GCase in vivo. For example, the cyclic guanidines of the invention may bind: (i) the catalytic site; (ii) an allosteric site; (iii), a site outside the catalytic site; and/or (d) a site outside an allosteric site, of GCase.

### III. Compositions (Including Pharmaceutical Compositions) Comprising the Cyclic Guanidines of the Invention

The cyclic guanidines of the invention may be synthetic or isolated from natural sources (such as yogurt). They may be comprised in compositions of any kind, and may be formulated as pharmaceutical compositions.

### Posology

The compounds of the present invention can be administered topically or by oral or parenteral routes, including intravenous, intramuscular, intraperitoneal, subcutaneous, transdermal, airway (aerosol), rectal, vaginal and topical (including buccal and sublingual) administration.

The amount administered can vary widely according to the particular dosage unit employed, the period of treatment, the age and sex of the patient treated, the nature and extent of the disorder treated, and the particular compound selected.

In general, the effective amount of the compound administered will generally range from about 0.01 mg/kg to 500 mg/kg daily. A unit dosage may contain from 0.05 to 500 mg of the compound and can be taken one or more times per day. The compound can be administered with a pharmaceutical carrier using conventional dosage unit forms either orally, parenterally, or topically, as described below.

The preferred route of administration is oral administration. In general, a suitable dose will be in the range of 0.01 to 500 mg per kilogram body weight of the recipient per day, preferably in the range of 0.1 to 50 mg per kilogram body weight per day and most preferably in the range 1 to 5 mg per kilogram body weight per day.

The desired dose is preferably presented as a single dose for daily administration. However, two, three, four, five or six or more sub-doses administered at appropriate intervals throughout the day may also be employed. These sub-doses may be administered in unit dosage forms, for example, containing 0.001 to 100 mg, preferably 0.01 to 10 mg, and most preferably 0.5 to 1.0 mg of active ingredient per unit dosage form.

### Formulation

The cyclic guanidine compound for use according to the invention may take any form. It may be synthetic or isolated from natural sources (for example from yogurt).

When isolated from a natural source, the compound may be purified. However, the compositions of the invention may take the form of foods (particularly yogurts, and more particularly the yogurts obtained or obtainable by the processes of the invention), as herein described. Such foods are preferably analysed to determine whether they meet a standard specification prior to use.

Compounds of the invention may be separated from the higher molecular weight components such as proteins and polysaccharides by extraction in polar solvents (such as ethanol/water mixtures, for example >50% v/v (e.g., up to ˜70% v/v) ethanol/water mixtures). Other suitable techniques include various membrane technologies. These include microfiltration, ultrafiltration and nanofiltration. Alternatively, or in addition, electrodialysis may also be used to concentrate the charged compound. These methods use membranes of pore sizes that allow only molecules below a certain size to pass or rely on charges on the molecules to allow or not allow them to pass through the membrane. Anion and cation exchange resins may also be used to concentrate the compounds.

When isolated from a natural source, the compound for use according to the invention may be purified. In embodiments where the compound is formulated together with a pharmaceutically acceptable excipient, any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc.

The pharmaceutical compositions may take any suitable form, and include for example tablets, elixirs, capsules, solutions, suspensions, powders, granules and aerosols.

The pharmaceutical composition may take the form of a kit of parts, which kit may comprise the composition of the invention together with instructions for use and/or a plurality of different components in unit dosage form.

Tablets for oral use may include the compound for use according to the invention, mixed with pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the compound for use according to the invention is mixed with a solid diluent, and soft gelatin capsules wherein the active ingredient is mixed with water or an oil such as peanut oil, liquid paraffin or olive oil.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate.

The compounds of the invention may also be presented as liposome formulations.

For oral administration the compound or compounds can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch.

In another embodiment, the compounds of the invention are tableted with conventional tablet bases such as lactose, sucrose, and cornstarch in combination with binders such as acacia, cornstarch, or gelatin, disintegrating agents intended to assist the break-up and dissolution of the tablet following administration such as potato starch, alginic acid, corn starch, and guar gum, lubricants intended to improve the flow of tablet granulations and to prevent the adhesion of tablet material to the surfaces of the tablet dies and punches, for example, talc, stearic acid, or magnesium, calcium, or zinc stearate, dyes, colouring agents, and flavouring agents intended to enhance the aesthetic qualities of the tablets and make them more acceptable to the patient.

Suitable excipients for use in oral liquid dosage forms include diluents such as water and alcohols, for example, ethanol, benzyl alcohol, and the polyethylene alcohols, either with or without the addition of a pharmaceutically acceptably surfactant, suspending agent or emulsifying agent.

The compounds of the invention may also be administered parenterally, that is, subcutaneously, intravenously, intramuscularly, or interperitoneally. In such embodiments, the compound is provided as injectable doses in a physiologically acceptable diluent together with a pharmaceutical carrier (which can be a sterile liquid or mixture of liquids). Suitable liquids include water, saline, aqueous dextrose and related sugar solutions, an alcohol (such as ethanol, isopropanol, or hexadecyl alcohol), glycols (such as propylene glycol or polyethylene glycol), glycerol ketals (such as 2,2-dimethyl-1,3-dioxolane-4-methanol), ethers (such as poly(ethylene-glycol) 400), an oil, a fatty acid, a fatty acid ester or glyceride, or an acetylated fatty acid glyceride with or without the addition of a pharmaceutically acceptable surfactant (such as a soap or a detergent), suspending agent (such as pectin, carbomers, methylcellulose, hydroxypropylmethylcellulose, or carboxymethylcellulose), or emulsifying agent and other commonly used pharmaceutical adjuvants.

Suitable oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate.

Suitable soaps include fatty alkali metal, ammonium, and triethanolamine salts and suitable detergents include cationic detergents, for example, dimethyl dialkyl ammonium halides, alkyl pyridinium halides, and alkylamines acetates; anionic detergents, for example, alkyl, aryl, and olefin sulphonates, alkyl, olefin, ether, and monoglyceride sulphates, and sulphosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention will typically contain from about 0.5 to about 25% by weight of the compound for use according to the invention in solution. Preservatives and buffers may also be used. In order to minimize or eliminate irritation at the site of injection, such compositions may contain a non-ionic surfactant having a hydrophile-lipophile balance (HLB) of from about 12 to about 17. The quantity of surfactant in such formulations ranges from about 5 to about 15% by weight. The surfactant can be a single component having the above HLB or can be a mixture of two or more components having the desired HLB. Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The compound or compounds for use according to the invention may also be administered topically, and when done so the carrier may suitably comprise a solution, ointment or gel base. The base, for example, may comprise one or more of the following: petrolatum, lanolin, polyethylene glycols, bee wax, mineral oil, diluents such as water and alcohol, and emulsifiers and stabilizers. Topical formulations may contain a concentration of the compound from about 0.1 to about 10% w/v (weight per unit volume).

When used adjunctively, the compound or compounds for use according to the invention may be formulated for use with one or more other drug(s). Thus, adjunctive use may be reflected in a specific unit dosage designed to be compatible (or to synergize) with the other drug(s), or in formulations in which the compound or compounds are admixed with one or more enzymes. Adjunctive uses may also be reflected in the composition of the pharmaceutical kits of the invention, in which the compounds of the invention are co-packaged (e.g., as part of an array of unit doses) with the enzymes. Adjunctive use may also be reflected in information and/or instructions relating to the co-administration of the compound or compounds and/or enzyme.

### IV. Medical and Health Implications and Applications of the Invention

### Glucocerebrosidase and Neurodegeneration

Glucocerebrosidase (GCase; EC 3.2.1.45), also referred to as GBA or acid β glucocerebrosidase, is encoded by the GBA1 gene. It is a lysosomal enzyme responsible for the conversion of the glycosphingolipid glucocerebroside (also called glucosylceramide) and glucosylsphingosine to glucose and ceramide.

GCase plays a key role in many neurodegenerative diseases, including Gaucher's disease (GD), Lewy body diseases (LBD), including Parkinson's disease (PD) and Lewy body dementia (LDD), Alzheimer's disease (AD) and other synucleinopathies. This is explained in more detail below.

### Parkinson's Disease and Lewy Body Dementia

Parkinson's disease (PD) is the second most common progressive neurodegenerative disorder, affecting about 1% to 5% of the population over the age of 60. Clinically, PD is associated with motor impairments including bradykinesia, akinesia, rigidity, resting tremor and gait disturbance. These symptoms are, in major part, due to the progressive loss of dopaminergic neurons in the substantia nigra pars compacta and the presence of Lewy bodies (LBs) in vulnerable populations of neurons. The LBs form intraneuronal cytoplasmic inclusions, composed of the misfolded, aggregated α-synuclein (α-syn) protein, together with other proteins and organelles.

Decreased GCase enzymatic activity is a well-known genetic risk factor for PD, and reduced activity has been observed in the substantia nigra of sporadic PD patients and correlated with the accumulation of α-syn (see Moors et al. (2019) Characterization of Brain Lysosomal Activities in GBA-Related and Sporadic Parkinson's Disease and Dementia with Lewy Bodies. Mol. Neurobiol, 56, 1344-1355, and Gundner et al. (2019) Path mediation analysis reveals GBA impacts Lewy body disease status by increasing α-synuclein levels. Neurobiol. Dis., 121, 205-213). Both genetic and post-mortem evidence from PD patients clearly highlight the key role of α-syn in PD pathology. Alpha-syn has also been shown to aggregate in other diseases, grouped under the term synucleinopathies. Targeting neuronal accumulation of α-syn is thus appealing to potentially halt or delay the progression of PD and other synucleinopathies.

Lewy body dementia (also known as Dementia with Lewy Bodies, DLB) shares many clinical, neurochemical and morphological features with PD, such that PD and DLB may be seen as extremes of a single spectrum disorder (α-synuclein-associated disease spectrum or Lewy body disease (LBD)). DLB with Lewy bodies is the second most common neurodegenerative dementia after AD. To date, no drug with disease modifying effects is available for DLB and treatment is aimed at trying to counterbalance the underlying neurotransmission disturbances (Jellinger (2020) How can we best care for people with dementia with Lewy bodies pharmacologically? Expert Opinion on Pharmacotherapy 21, 513-515).

The major protein component of Lewy bodies is α-synuclein (α-syn or SNCA), a synaptic protein with the propensity to misfold and aggregate, leading to PD or DLB. Mutations in the gene encoding GBA have been identified as the most common known genetic risk factor for the development of PD. Reduction of GCase activity (e.g., as a result of mutations in GBA1) causes accumulation of glycosphingolipids that contribute to the pathology of PD and LBD, possibly via altering lysosomal function and exacerbation of α-synuclein (α-syn) aggregation through inhibition of autophagy via PPP2A inactivation. Numerous hypotheses have been advanced on the possible ways by which a reduction in GCase activity might induce α-syn accumulation, but none fully explain experimental data. It is likely that multiple mechanisms have a role and that different GBA1 mutations may preferentially lead to different pathogenic pathways (Toffoli et al. (2020) The biochemical basis of interactions between glucocerebrosidase and alpha-synuclein in GBA1 mutation carriers. J. Neurochem. 154(1):11-24).

Accumulating evidence over the past decade has also revealed an association between mutations in GBA and the development of PD and LBD. Clinical studies have reported parkinsonism among relatives of patients with Gaucher's disease (see below), while genotyping studies have demonstrated a higher incidence of GBA mutations in cohorts of PD patients of different ethnic origins, particularly those with early onset PD. Post-mortem examination of PD brains with GBA mutations revealed reduced levels of GBA protein, primarily in the substantia nigra. GBA knockdown in neurons led to decreased lysosomal degradation capacity, and increased levels of SNCA protein.

Risk factors for PD and LBD include advanced age (for example, humans of 60 years or older), a family history of PD, a high-risk genetic test profile (e.g., a GBA1 gene mutation), a high-risk biomarker profile (e.g., decreased levels of GCase protein or enzymic activity), and/or one or more non-specific neurological impairment(s) (including, for example, one or more of cognitive impairment, memory loss, depression, insomnia, tremor, anosmia, drooling, bradykinesia, hypokinesia, dizziness, fainting, dyskinesia, postural instability, rigidity, postural changes, facial masking and anxiety). Subjects at high risk of PD or LBD therefore include those having one or more of the foregoing risk factors, and the invention therefore finds application in the prevention of PD or LBD in such subjects.

GBA1 mutations are considered the most common genetic risk factor for PD (Palermo et al. (2019) Expanding GBA involvement in neurodegeneration. J Mol and Gen Med 13:4), and PD and DLB patients carrying a GBA1 mutation have earlier age of onset, suffer more frequently from cognitive decline and show more rapid disease progression (Lerche et al. (2020) PD:GBA1 mutation severity is associated with alpha synuclein profiles. ‘Movement Disorders 35 (3)).

The findings discussed above all point to GCase as a highly relevant therapeutic target to increase α-syn clearance and decrease α-syn pathological aggregation (Do et al. (2019) Glucocerebrosidase and its relevance to Parkinsons disease. Mol Neurodegen. 14, 36). For example, Ambroxol hydrochloride, a safe FDA-approved molecule, was proven to enhance GCase activity and to increase α-syn clearance (Ambrosi et al. (2015) Ambroxol-induced rescue of defective glucocerebrosidase is associated with increased LIMP-2 and saposin C levels in GBA1 mutant Parkinson's disease cells. Neurobiol. Dis. 2015, 82, 235-242). Wild type and α-syn transgenic mice treated with Ambroxol presented increased brain activity of GCase, associated with decreased levels of total and phosphorylated α-syn protein in different brain regions of the transgenic model. Based on the results obtained after Ambroxol treatment on GD patients and on PD rodent models, a pilot study in humans has been launched recently in order to determine the effective dose and to prove the efficacy of such a strategy on seventy-five PD patients (Silveira et al. (2019) Ambroxol as a novel disease-modifying treatment for Parkinson's disease dementia: Protocol for a single-centre, randomized, double-blind, placebo-controlled trial. BMC Neurol. 2019, 19, 20).

In parallel, another recent clinical trial enrolling seventeen PD patients showed Ambroxol crossed the BBB and increased the GCase activity in patients both with and without GBA1 mutation.

### Alzheimer's Disease

Alzheimer's disease (AD) is a progressive neurologic disorder that causes the brain to shrink (atrophy) and brain cells to die. AD is the most common cause of dementia (a continuous decline in thinking, behavioural and social skills that affects a person's ability to function independently). Early signs of the disease include short term memory loss. As the disease progresses, more severe memory impairments appear and the ability to carry out everyday tasks is lost. Medications are available which may temporarily improve or slow progression of symptoms, but there is a pressing need for new forms of treatment and prophylaxis.

GCase plays an important role in the pathogenesis of AD-GCase protein levels and enzyme activity are significantly decreased in sporadic AD. Thus, a deficiency of GCase is involved in progression of AD pathology and promotion of GCase activity is a therapeutic option for the treatment of AD (see Choi et al. (2015) Lysosomal Enzyme Glucocerebrosidase Protects against Aβ1-42 Oligomer-Induced Neurotoxicity. PloS ONE 10(12): e0143854).

### Synucleinopathies

Synucleinopathies comprise a diverse group of neurodegenerative diseases characterized by the presence of lesions composed of aggregates of conformational and posttranslational modifications of α-synuclein in certain populations of neurons and glia. Abnormal filamentous aggregates of misfolded α-synuclein protein are the major components of Lewy bodies, dystrophic (Lewy) neurites, and the Papp-Lantos filaments in oligodendroglia and neurons in multiple system atrophy linked to degeneration of affected brain regions. In contrast to the extracellular amyloid plaques found in the brains of Alzheimer's patients, Lewy bodies are intracellular.

The synucleinopathies include Lewy body diseases (LBDs), dementia with Lewy bodies, multiple system atrophy (MSA), Hallervorden-Spatz disease, Parkinson's disease (PD), the Lewy body variant of Alzheimer's disease (LBVAD), neurodegeneration with brain iron accumulation type-1 (NBIA-1), pure autonomic failure, neuroaxonal dystrophy, amytrophic lateral sclerosis and Pick disease and various tauopathies.

Thus, the invention finds application in the treatment or prophylaxis of various synucleinopathies including LBD, PD, DLB, multiple system atrophy (MSA), Hallervorden Spatz disease, Lewy body variant of Alzheimer's disease (LBVAD), neurodegeneration with brain iron accumulation type-1 (NBIA-1), pure autonomic failure, neuroaxonal dystrophy, amytrophic lateral sclerosis and Pick disease and various tauopathies.

### Tauopathies

The compounds of the invention find application in the treatment or prophylaxis of tauopathy. The tauopathies are a group of diverse dementias and movement disorders which have as a common pathological feature the presence of intracellular accumulations of abnormal filaments of tau protein. Examples include Down's Syndrome (DS), Corticobasal Degeneration (CBD), Frontotemporal Dementia with Parkinsonism linked to Chromosome 17 (FTDP17), Pick Disease (PiD) and Progressive Supranuclear Palsy (PSP).

### Gaucher's Disease

Gaucher's Disease (GD) is an autosomal recessive lysosomal storage disorder that results from loss-of-function mutations in GBA1 and an attendant deficiency of GCase activity. GD is one of the more common lysosomal storage disorders, occurring with an incidence of approximately 1 in 50,000 to 100,000 live births.

GCase deficiency in GD leads to the accumulation of glucocerebroside in tissue macrophages, affecting the haematological, visceral, bone and neurologic systems. Gaucher disease is classified into three broad phenotypes based upon the presence or absence of neurological involvement: type 1 (non-neuronopathic), type 2 (acute neuronopathic), and type 3 (subacute neuronopathic). While enzyme replacement is effective in managing the visceral disease, treatment of the neurological manifestations has proved to be more challenging.

Loss-of-function mutations in GBA1 are also a key genetic risk factor for the α-synucleinopathies, including PD and DLB (see Blandini et al. (2019) Glucocerebrosidase mutations and synucleinopathies: toward a model of precision. Medicine. Mov. Disord. 34 (1), 9-21).

In a zebrafish model of GD, early microglial activation with marked neuroinflammation in association with a marked increase of transcript levels of a master regulator of inflammation (miR-155) has been observed. miR-155 has been implicated in a wide range of different neurodegenerative disorders (Watson et al. (2019) Ablation of the pro-Inflammatory master regulator miR-155 does not mitigate neuroinflammation or Neurodegeneration in a vertebrate model of Gaucher's disease. Neurobiol. Dis. 127, 563-569).

The iminosugar isofagomine (IGF) has been shown to increase GCase activity in vitro and in mice in a tissue-specific manner (Sun et al. (2012) Ex vivo and in vivo effects of isofagomine on acid beta-glucosidase variants and substrate levels in Gaucher disease. J. Biol. Chem. 287, 4275-4287). However, IGF also exhibits an undesirable glycogen phosphorylase inhibitory activity.

### GCase Mediated Neuroprotection

In the light of the involvement of GCase deficiency in neurodegenerative disorders (as explained above), the invention finds broad application in neuroprotection.

More particularly, the invention may be used in the treatment or prevention of GCase deficiency, for example in the treatment or prevention of neurodegeneration caused by GCase deficiency and other diseases mediated (at least in part) by GBA deficiency (referenced herein as GBA deficiency diseases).

The invention therefore finds application in the treatment or prevention of LBD (including PD and DLB), AD and GD.

### Effect on Microbiota

Methanogenic archaea are involved in periodontitis in humans and have recently been implicated in obesity and digestive tract disorders. These microorganisms are broadly resistant to antibiotics, except for metronidazole and ornidazole (see Khelaifia et al. (2013) Hydrophobicity of imidazole derivatives correlates with improved activity against human methanogenic archaea. International Journal of Antimicrobial Agents 41, 544-547). The cyclic guanidines of the invention may inhibit methanogenic archaea in the digestive tract, and so find application in the treatment of methanogenic archaea-related infections, obesity, periodontitis and other digestive tract disorders. They may also find application in reducing methane production by ruminants and thereby reducing the release of greenhouse gases and improve ruminant feed efficiency (see Huws et al. (2018) Addressing Global Ruminant Agricultural Challenges Through Understanding the Rumen Microbiome: Past, Present, and Future. Front Microbiol., 9, 2161).

Thus, the processes of the invention (see the following section) also find application in the production of improved yogurts and yogurt-based and yogurt-derived foods and beverages (which may contain yogurt or yogurt fractions) and yogurt-based and yogurt-derived animal feeds (which may also contain yogurt or yogurt fractions), permitting anti-archaeal functional properties of yogurt and animal feeds to be verified and optimized via appropriate quality control and process design. For example, the invention contemplates animal feeds (e.g., ruminant feeds) which contain the cyclic guanidine or compositions of the invention. In such embodiments, the cyclic guanidine may take any form, being for example as described in section II (above). In preferred such embodiments, the cyclic guanidine takes the form of a yogurt or yogurt fraction (see, e.g., section III, above).

### V. Yoghurt Production, Processing and Quality Control

The discovery of bioactive cyclic guanidine biomolecules which promote GCase activity as bioactive principles in yogurt finds application in the production of improved yogurts and yogurt-based and yogurt-derived foods and beverages (which may contain yogurt or yogurt fractions), since it permits functional properties of yogurt to be verified and optimized via appropriate quality control and process design. Yogurt-derived foods and beverages include those comprising yogurt extracts, for example extracts obtained with polar solvents (such as ethanol). Yogurt-based foods and beverages include those containing (e.g., being supplemented with) yogurt (e.g., dried yogurt).

For example, the invention contemplates processes for the production of yogurts, or for monitoring the quality of a yogurt, comprising the steps of: (a) providing a yogurt sample; and (b) testing the sample for GCase activity or for the presence of glycosidase modulatory activity, for example for the inhibition or promotion of one or more glycosidases.

The invention also contemplates a process for producing a yogurt, or for monitoring the quality of a yogurt, comprising the steps of: (a) providing a yogurt sample; and (b) testing the sample for the presence or absence of a cyclic guanidine, or to measure the amount of a cyclic guanidine in the sample.

Also contemplated is a process for producing a yogurt, or for monitoring the quality of a yogurt, comprising the steps of: (a) providing a yogurt sample; and (b) testing the sample for the presence of glycosidase modulatory activity, for example for the inhibition or promotion of one or more glycosidases.

Those skilled in the art will be able to select any of a wide range of suitable GCase assays for use in the processes of the invention.

Thus, the invention exploits the more general discovery that yogurts contain other bioactive principles which modulate glycosidases, with some glycosidase enzymes inhibited and others promoted. Thus, it permits further optimization of the functional properties of yogurt by testing for glycosidase modulation.

In such processes, the glycosidase may be selected from the following glycosidase classes: α-glucosidases; ß-glucosidases; α-galactosidases; ß-galactosidases; α-mannosidases; α-fucosidases; α-iduronidases; β-glucuronidases; β-mannosidases; hexosaminidases; α-N-acetylglucosaminidases; α-N-acetylgalactosaminidases; β-N-acetylglucosaminidases; β-N-acetylgalactosaminidases; sialidases; heparinases; glucocerebrosidases; neuraminidases; hyaluronidase; amylases; and two or more of the foregoing enzyme classes.

More preferably, the glycosidase may be selected from the following glycosidase classes: α-glucosidases; ß-glucosidases; α-galactosidases; ß-galactosidases; α-mannosidases; β-mannosidases; β-glucuronidases; α-N-acetylglucosaminidases; α-N-acetylgalactosaminidases; β-N-acetylglucosaminidases; β-N-acetylgalactosaminidases; and two or more of the foregoing enzyme classes. In some embodiments of this aspect of the invention, the yogurt sample is tested for inhibition of β-mannosidase activity. This may be particularly preferred in applications involving the treatment or prevention of cancer, when β-mannosidase inhibiting compositions derived from yogurt (or improved yogurts in which the content of ß-mannosidase inhibitory factor is maximized and/or monitored during production) may be produced (see section VI, below).

In all cases, the testing step(s) may be preceded by an extraction step in which the yogurt sample is extracted to yield a fraction enriched in polar chemicals (such as amino acids and iminosugar-like compounds). The use of an extraction step increases the sensitivity and reliability of any GCase assay, while facilitating the detection of any cyclic guanidines, and so is a preferred element of the processes of the invention described above.

Thus, the invention contemplates a process for producing a yogurt comprising the steps of: (a) providing a milk product; (b) fermenting the milk product, for example with bacteria comprising Lactobacillus delbrueckii subsp. bulgaricus, to yield a yogurt; (c) providing a sample of the yogurt of step (b); and (d) testing the sample: (i) for GCase activity; and/or (ii) for the presence or absence of a cyclic guanidine; and/or (iii) to measure the amount of a cyclic guanidine in the sample; and/or (iv) for the presence of glycosidase modulatory activity, for example for the inhibition or promotion of one or more glycosidases.

### Samples

The yogurt sample used in the processes of the present invention may take any convenient form. For example, it may take the form of an aliquot of a production batch of the yogurt.

Alternatively, the samples may be pre-processed in any of a wide variety of ways prior to use. Pre-processing may involve physical or chemical pre-processing, for example powdering, grinding, freezing, evaporation, homogenization, filtration, dilution, pressing, spray drying, freeze-drying and/or extrusion.

### Extraction

The optional extraction step may take any convenient form, but preferred is extraction in a polar solvent followed by ion exchange chromatography. For example, a yogurt sample may be extracted by mixing with 90% aqueous ethanol to yield a supernatant fraction. The supernatant fraction (which is enriched in polar chemicals, such as amino acids, iminosugar-like compounds and the cyclic guanidines of the invention) is then filtered to yield a polar fraction. The polar fraction is then contacted with an acidic cation exchange resin, and the polar chemicals eluted (for example, with a 2M ammonia solution) after washing (e.g., with water).

Where chromatography is employed, those skilled in the art, by routine trial and error and by using common general knowledge, will be able readily to determine the appropriate resin and/or column characteristics according to the circumstances, including inter alia the extract under study and the nature of the solvent used in the extraction and the nature of the cyclic guanidine expected in those solvents. Chromatographic fractionation preferably comprises ion exchange chromatography. Ion-exchange chromatography partially purifies ionic species to concentrate them and remove contaminating substances. Those skilled in the art, by routine trial and error and using common general knowledge, will be able readily to identify suitable column packing materials and mobile phase(s), which will depend inter alia on the quantities to be fractionated, the extracts under study and the nature of the solvent used in the extraction. Particularly preferred in the methods of the present invention are strongly acidic cation exchange resins which can be used in either the free acid or hydrogen (H⁺) form or in the ammonium (NH₄⁺) salt form). These forms adsorb cations from solution and release an equivalent number of counter-ions back into solution (either H⁺ or NH₄⁺ ions, depending on the form used).

Suitable polar solvents for use in the process of the invention include without limitation organic solvents such as organic alcohols. Preferred are ethanol and methanol, as well as ethanol/water or methanol/water mixtures. Preferably, the polar solvent is selected from 50 to 90% ethanol/water, 31 to 50% ethanol/water, and up to 30% ethanol/water. Particularly preferred is a polar solvent which is approximately 90% ethanol/water. The conditions (time, temperature, degree of agitation etc.) under which the extraction(s) are performed can be readily determined empirically and vary according to the nature of the sample, the nature of any pre-processing and the solvent system selected.

### Cyclic Guanidine Analysis

In cases where the process comprises the step of testing the sample for the presence or absence of a cyclic guanidine, or to measure the amount of a cyclic guanidine in the sample, the cyclic guanidine is preferably as described in section II (above).

Those skilled in the art will be able to select any of a wide range of suitable analytical techniques for use in testing the sample for the presence or absence of a cyclic guanidine, or to measure the amount of a cyclic guanidine in the sample. Such techniques include, without limitation: (a) nuclear magnetic resonance (NMR); and/or (b) spectral analysis.

Spectral analysis is particularly preferred, and may produce any or all of the following spectra: (a) mass spectra (e.g., the mass to charge (m/z) value versus abundance), and/or (b) chromatographic data (e.g., spectra, column retention times, elution profiles etc), and/or (c) photodiode array (PDA) spectra (e.g., in both UV and visible ranges), and/or (d) electrochemical detection; and/or (e) nuclear magnetic resonance (NMR) spectra (e.g., spectral data sets obtained via ¹H and/or ¹³C NMR).

When used according to the invention, the spectral analysis may be coupled with fractionation of the sample, for example by use of GC-MS and/or HPLC-PDA-MS. Other techniques include hydrophilic interaction liquid chromatography (HILIC).

### VI. Other Yogurt Bioactives

As explained above, it has been discovered that yogurt contains factors which modulate a range of different glycosidases. Of particular importance is the discovery of a ß-mannosidase inhibitory factor. This enzyme is elevated in certain cancers, including oesophageal squamous cell carcinoma. This is one of the most aggressive upper aerodigestive tract malignancies and major cause of cancer-related mortality worldwide. The invention therefore finds application in the treatment or prevention of cancer, using ß-mannosidase inhibiting compositions derived from yogurt (or by producing improved yogurts in which the content of ß-mannosidase inhibitory factor is maximized and/or monitored during production).

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1: Yogurt Increases the Activity of β-glucocerebrosidase

### Background

In a study to determine if the claimed health benefits of yogurts were due to compounds produced by the lactic acid bacteria rather than the organisms themselves colonizing the intestine, cultures of yogurt were processed using cation and anion exchange resins and then assayed against a panel of glycosidase enzymes. Glycosidases have many important functions in the body and their activities are often perturbed in most if not all illnesses (Dwek, R. A. Chem Rev. 1996, 28, 683-720; Reily, C. et al., Nature Reviews Nephrology, 2019, 15, 346-366). Surprisingly it was noted that some glycosidases were inhibited, and others promoted but not equally by all yogurt brands studied. The most active brand was one from Bulgaria (Na Baba) and we conclude that compounds produced by the Lactobacillus species or varieties present may be responsible for the different activities observed.

### Methods

Preparation of yogurt for assays. A sample of 200 mL of Na baba yogurt from Bulgaria (L 01/02/15, 4.5%) was extracted in 90% aq. ethanol with mixing for 15 hours and after filtering the supernatant, the amino acids and iminosugar-like compounds were retained on strongly acidic cation exchange resin (Amberlite IR120 H⁺ form), washed with copious water and then displaced with 2M ammonia solution (75 mg yield). This fraction was tested on a panel of glycosidases. Recultured Na baba and other yogurts purchased in the UK were processed in the same way e.g., Dale Farm (Natural Greek Style LL40100 use by 5 Feb. 2018), Tesco (Greek Style Natural use by 9 Nov. 2018), Rachels (Organic Greek Style Bio-live use by 29 Jun. 2019) and Actimel Original. The cation exchange fraction was further processed by running through a strongly basic anion exchange resin (CG400 OH⁻ form) (Na baba yield 30 mg unretained) and retained material (Na baba 35 mg) was displaced with 1 M HOAc. Recultured Na Baba gave the same result.

Glycosidase assays. Most enzymes and para-nitrophenyl substrates were purchased from Sigma with the exception of human recombinant β-glucocerebrosidase (Cerezyme) obtained from Genzyme, Europe. Pure iminosugars which inhibit β-glucosidases (DMDP and calystegine B₂) were used as controls. Yogurt samples were tested at 1 mg/ml. Enzymes were assayed at 27° C. in 0.1 M citric acid/0.2 M disodium hydrogen phosphate buffers at the optimum pH for the enzyme (pH 5.9 for Cerezyme). The incubation mixture consisted of 10 μL enzyme solution, 10 μL of 1 mg/mL aqueous inhibitor solution and 50 μL of the appropriate 5 mM para-nitrophenyl substrate made up in buffer at the optimum pH for the enzyme. Cerezyme was assayed against para-nitrophenyl (PNP) β-glucopyranoside. The reactions were stopped by addition of 70 μL 0.4 M glycine (pH 10.4) during the exponential phase of the reaction, which had been determined at the beginning using uninhibited assays in which water replaced inhibitor. Final absorbances were read at 405 nm using a Versamax microplate reader (Molecular Devices). Assays were performed in triplicate and the mean percentage inhibitions used. A negative value indicates enzyme activation.

### Results

The yogurts showed activities against a number of the glycosidases with some enzymes inhibited and others promoted. Most notable of the inhibitions were α- and β-galactosidase and β-mannosidase and rat intestinal α-glucosidase while other α- and β-glucosidases were generally promoted with Dale Farm giving 150% promotion of a Bacillus α-glucosidase. The differences in glycosidase results between the yogurts were high but promotion of the β-glucocerebrosidase was shown by most yogurts but not all (e.g., Rachels Natural Organic not shown in the table which gave no promotions or inhibitions). The degree of promotion of the activity did vary with Na Baba giving very high activity in the unretained fraction from anion exchange resin in the OH— form. Reference iminosugars gave the expected inhibition of the β-glucocerebrosidase and other glycosidases. The inhibition of β-mannosidase by Na Baba was also of interest because this enzyme is elevated in certain cancers including oesophageal squamous cell carcinoma which is one of the most aggressive upper aerodigestive tract malignancies and major cause of cancer-related mortality worldwide (as explained above).

Table 1 below shows the glycosidase assay results expressed as % inhibition.

| | | α-D- | α-D- | β-D- | α-D- | β-D- | glucocerebrosidase |
|---|---|---|---|---|---|---|---|
| | | glucosidase | glucosidase | glucosidase | galactosidase | galactosidase | (GC) |
| Yogurt or compound | Other details | Yeast | Bacillus | almond | Green coffee | bovine liver | Genzyme |
| Na baba, Bulgaria | cation resin retained | −30 | −27 | 17 | 19 | 73 | −72 |
| Na baba | anion resin retained | −11 | −37 | 14 | 7 | 31 | −28 |
| Na baba | anion resin unretained | −10 | 17 | 11 | 41 | 15 | −330 |
| Tesco, UK | Greek Style | | | | | | −154 |
| Dale Farm, UK | United Feeds | −28 | −158 | −4 | | −10 | −54 |
| Activia UK | Original | −13 | −27 | −31 | 14 | 20 | ND |
| iminosugar reference | Calystegine B2 0.8 mM | 18 | −5 | 96 | 99 | 44 | 100 |
| iminosugar reference | DMDP 0.8 mM | 100 | 93 | 97 | 7 | 99 | 44 |

| | | α-D- | β-D- | N-acetyl- | N-acetyl- | β- | α-D- |
|---|---|---|---|---|---|---|---|
| | | mannosidase | mannosidase | β-D gluc | β-D-gluc | glucuronidase | glucosidase |
| Yogurt or compound | Other details | Jack bean | C. fimi | Bovine kidney | Jack bean | bovine liver | rat intestine |
| Na baba, Bulgaria | cation resin retained | 12 | 56 | 0 | −27 | 3 | 5 |
| Na baba | anion resin retained | | | −36 | −68 | 15 | 70 |
| Na baba | anion resin unretained | | | 0 | 2 | −3 | 46 |
| Tesco, UK | Greek Style | | | | | | |
| Dale Farm, UK | United Feeds | | | −15 | | 14 | 15 |
| Activia UK | Original | 44 | 0 | −24 | −30 | | 0 |
| iminosugar reference | Calystegine B2 0.8 mM | 8 | −5 | 0 | 0 | | 22 |
| iminosugar reference | DMDP 0.8 mM | 3 | 93 | −7 | −7 | | 77 |

### CONCLUSION

The cation exchange resin retained fractions of yogurts can give pronounced inhibitions and promotions of glycosidases. The promotions of bacterial glucosidases could be responsible for some of the claims of positive effects of yogurts on digestion. Inhibitions of β-glucuronidase could give benefits for toxin removal from the body with many excreted as glucuronides which will be excreted more efficiently with lower serum and digestive tract β-glucuronidase activity. It may also be significant for reducing urinary tract infections as certain pathogens of the urinary tract such as E. coli require β-glucuronidase activity to degrade and adhere to mucopolysaccharides (Chang et al., 1989, Appl. Environ. Microbiol. 55:335-339). The enzyme's activity level in body fluids is also deemed a potential biomarker for the diagnosis of some pathological conditions. Moreover, due to its role in colon carcinogenesis and certain drug-induced dose-limiting toxicities, the development of potent inhibitors of β-glucuronidases in human intestinal microbiota has aroused increased attention over the years. It has been reported previously that various Lactobacillus species can reduce intestinal β-glucuronidases, but the active components were not identified (Han, S Y., Huh, C S., Ahn, Y T. et al. (2005) Hepatoprotective effect of lactic acid bacteria, inhibitors of β-glucuronidase production against intestinal microflora. Arch Pharm Res 28, 325). These lactic acid bacteria also showed a potent hepatoprotective effect.

The inhibition of the β-galactosidase (lactase) is somewhat curious as it would suggest yogurts might protect lactose and therefore make lactose intolerance worse although yogurts are thought to be suitable for people with lactose intolerance. This finding indicates that the invention may also find application in processes for the production and monitoring of yogurts which comprise the step of determining whether the yogurt contains lactase inhibiting factors, since significant differences in this regard were observed (e.g., the Dale Farm yogurt promoted the enzyme activity).

### Example 2: Identification of Bioactive Guanidines in Yogurt

### Background

Cation exchange resin retained compounds from yogurts had been found to greatly promote β-glucocerebrosidase activity in vitro. The activity was found to be unretained on strongly acidic anion exchange resin. The active fractions were analysed by gas chromatography mass spectrometry (GCMS) and by NMR. Predictions were made about the structure of the active compound, and this was synthesised and included in enzyme assays.

### Methods

Gas chromatography-mass spectrometry (GC-MS). Samples (0.5 mg) of synthetic compound or yogurt fractions were reacted with 30 μL of Pierce TriSil reagent; after 20 minutes the trimethylsilylated (TMS) compounds were analysed on a Perkin Elmer Turbomass spectrometer using a high polarity fused-silica column (Varian ‘Factor Four’ VF-5 ms column, 25 m×0.25 mm i.d., 0.25 mm phase thickness). The carrier gas (helium) flow rate 1 mL min⁻¹. The TMS-derivatives were separated using a temperature program starting at 160° C. for 5 min, followed by a linear increase to 300° C. at a rate of 10° C. min−1. Electron impact mass spectrometry of the column eluant was carried out with the quadrupole ion filter system run at 250° C. constantly during analysis.

Glycosidase Assays. β-glucocerebrosidase enzyme (Human recombinant imiglucerase, obtained from Genzyme, Europe) were carried at 27° C. in 0.1 M citric acid/0.2 M disodium hydrogen phosphate buffer at pH 5.9. The incubation mixture consisted of 10 μL of enzyme solution, 10 μL of aqueous solution of extract or compound at the required concentration and 50 μL of 5 mM para-nitrophenyl (PNP) β-glucopyranoside (Sigma-Aldrich) made up in buffer at pH 5.9. The reactions were stopped by addition of 70 μL 0.4 M glycine (pH 10.4) during the exponential phase of the reaction, which had been determined at the beginning using uninhibited assays in which water replaced inhibitor. Final absorbances were read at 405 nm using a Versamax microplate reader (Molecular Devices). Assays were carried out in triplicate and the values given are means. Results are shown as % inhibition where a negative value indicates enzyme activation.

### Results

Proton NMR analysis of the active Na Baba fraction from anion exchange resin gave a simple spectrum suggesting a structure as shown below of (S)-2-imino-3-methylimidazolidine-4-carboxylic acid (1).

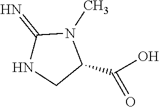

The structure would also fit the binding to a cation exchange resin but not an anion exchange resin in the OH⁻ form which tend to retain strongly acidic molecules. GCMS of the compound as a tms derivative gave a distinctive peak at 6.20 minutes with fragments at 155 (30%), 171 (100%) and the molecular ion (di-tms) was seen at 288 (2%). The yogurts gave a peak at 6.0 minutes with distinctive fragments 171 (100% and 256 (50%) with no clear molecular ion.

In assays on β-glucocerebrosidase the synthesised predicted structure (1) gave strong promotion equalling that of the yogurt purified compound but gave different activity against other glycosidases. Reference iminosugars gave the expected inhibition of the same enzyme. Table 2 below shows the glycosidase data expressed as % inhibition.

| | | | | | N-acetyl- |
|---|---|---|---|---|---|
| | | α-D- | β-D- | glucocerebrosidase | β-D-gluc |
| Yogurt or | | glucosidase | glucosidase | (GC) | Bovine |
| compound | Other details | Yeast | almond | Genzyme | kidney |
| Na baba | anion resin unretained | −10 | 11 | −330 | 0 |
| 1 | 8 mM except GC | 44 | 20 | −390 | 34 |
| | 0.8 mM | | | | |
| reference | calystegine B2 | 18 | 96 | 100 | 0 |
| iminosugar | 0.8 mM | | | | |
| reference | DMDP 0.8 mM | 100 | 97 | 44 | −7 |
| iminosugar | | | | | |

### CONCLUSION

The predicted yogurt compound was produced and showed equal strong promotion of ß-glucocerebrosidase to yogurt fractions in vitro. However, the GC mass spectrum and assay results are not exactly the same and so we assume the active yogurt molecule might be an isomer of (S)-2-imino-3-methylimidazolidine-4-carboxylic acid. These are likely to be new natural products.

### Example 3: NMR Analysis of Compound Extracted from Na Baba Yogurt

Heteronuclear multiple bond correlation (hmbc) yields correlations between carbons and protons that are separated by two or three bonds. Due to the weak sample proton attachment to individual carbon atoms were assigned from HMQC spectra. No COSY correlations were identified probably due to the weakness of the extract.

| | Proton | Carbon |
|---|---|---|
| | chemical shift | chemical shift |
| Me | 2.63 | 36 |
| | (3H, broad s, Me) | |
| CH | 3.50 | 53 |
| | (1H, m, CH) | |
| CH₂ | 2.85 | 54 |
| | (2H, m, CH₂) | |
| C═N | | 159 |
| C═O | | 175 |

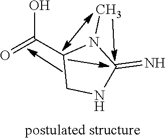

### EXPERIMENTAL

¹H NMR (500 MHz, D₂O) δ/ppm 2.63 (3H, broad s, Me), 3.50 (1H, m, CH), 2.85 (2H, m, CH₂)

¹³C NMR (126 MHz, D₂O) δ/ppm 175.2 (C═O), 159.0 (C═N), 54.0 (CH₂), 53.0 (CH), 36.1 (CH₃)

The singlet at 2.63 ppm in the proton spectra is postulated to be a methyl attached to nitrogen. This shows hmbc correlations to signals at 159 ppm and 53 ppm implying the methylated nitrogen is attached to the CH at 3.50 ppm in the proton spectra and a C═N. Further hmbc correlations from the CH at 3.50 ppm in the proton spectra to signals at 175 ppm, 159 ppm and 36 ppm indicate the CH to be attached to a carbonyl and support the postulated structure.

Since the CH appears as a multiplet it must be attached to the methylene at 2.85 ppm in the proton spectra although no COSY correlations could be detected and no hmbc correlations from the methylene.

Based on these interpretations the structure is postulated to be as described above. The chemical shifts of the protons are in good agreement with those expected from standard tables of substituent effects, and the carbon shifts match closely the predicted values (ACD CNMR predictor).

### Example 4: Commercial Potential Yogurt Components not Causing Promotion of GCase

### Background

Cation exchange resin retained compounds from certain yogurts had been found to greatly promote β-glucocerebrosidase activity in vitro. The activity was found to be unretained on strongly acidic anion exchange resin. The active fractions were analysed by gas chromatography mass spectrometry (GCMS) and by NMR. The postulated active compound ((S)-2-imino-3-methylimidazolidine-4-carboxylic acid, see Example 3) was synthesized. It was included in enzyme assays and was effective at promoting the enzyme activity in vitro. The assay was also extended to various other commercially available small natural molecules (which may also be present in yogurt), and the results are set out below.

### Materials

Pure compounds as listed in the results table below were purchased from Sigma-Aldrich.

### Glycosidase Assays

β-glucocerebrosidase enzyme (Human recombinant imiglucerase, obtained from Genzyme, Europe) were carried at 27° C. in 0.1 M citric acid/0.2 M disodium hydrogen phosphate buffer at pH 5.9. The incubation mixture consisted of 10 μL of enzyme solution, 10 μL of compound at the concentration shown in the results table and 50 μL of 5 mM para-nitrophenyl (PNP) β-glucopyranoside (Sigma-Aldrich) made up in buffer at pH 5.9. The reactions were stopped by addition of 70 μL 0.4 M glycine (pH 10.4) during the exponential phase of the reaction, which had been determined at the beginning using uninhibited assays in which water replaced inhibitor. Final absorbances were read at 405 nm using a Versamax microplate reader (Molecular Devices). Assays were carried out in triplicate, and the values given are means of the three replicates per assay. A negative value indicates enzyme activation.

### Results

### Table Showing β-Glucocerebrosidase Results for Pure Commercial Compounds Expressed as % Inhibition

| Compound | Conc | Cerezyme |
|---|---|---|
| adenine | 10 mg/ml | −22 |
| betaine | 10 mg/ml | 0.1 |
| caffeic acid | 5 mg/ml | −1.8 |
| chlorogenic acid | 5 mg/ml | 57.1 |
| cytosine | 10 mg/ml | −1.6 |
| nicotinamide | 5 mg/ml | −11.3 |
| nicotinic acid | 5 mg/ml | −12.7 |
| 6-OH-nicotinic acid | 5 mg/ml | −1.4 |
| quercetin | 5 mg/ml | 39.4 |
| trigonelline | 1 mg/ml | 3.1 |
| trigonelline | 5 mg/ml | −12.6 |
| 6-OH-trigonelline | 5 mg/ml | 8.6 |
| tryptamine | 5 mg/ml | 42.2 |
| uracil | 5 mg/ml | 4.5 |

Most of the compounds tried had little activity and some inhibited the enzyme, e.g., chlorogenic acid, tryptamine and quercetin. Adenine had some promotion activity but weaker than that seen with the yogurts and the postulated active compound ((S)-2-imino-3-methylimidazolidine-4-carboxylic acid.

### CONCLUSION

Commercially available small molecules that might be present in yogurts showed no great effect on the activity of the β-glucocerebrosidase in vitro.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A composition comprising a cyclic guanidine for use in a method of neuroprotection or for treating or preventing a GBA deficiency disease.

2. The composition for use according to claim 1, wherein the cyclic guanidine is a glucocerebrosidase activator.

3. The composition for use according to claim 2, wherein the cyclic guanidine glucocerebrosidase activator increases the catalytic activity of human ß-glucocerebrosidase >20%, e.g., >50%, in vitro in 0.1 M citric acid/0.2 M disodium hydrogen phosphate buffer at 27° C. and pH 5.9.

4. The composition for use according to claim 1, wherein the cyclic guanidine has the formula I: 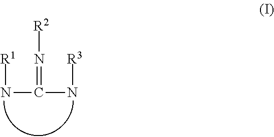
or a pharmaceutically acceptable salt, derivative, solvate, isomer, tautomer, N-oxide, ester, prodrug, isotope or protected form thereof, wherein R¹, R² and R³ are each independently selected from: H, methyl and optionally substituted C1-6 alkyl, C1-6 alkenyl or C1-6 alkynyl and wherein the connecting semi-circular line indicates one or more linking atoms/groups.

5. The composition for use according to claim 1, wherein the cyclic guanidine has the formula II: 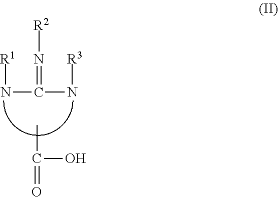
or a pharmaceutically acceptable salt, derivative, solvate, isomer, tautomer, N-oxide, ester, prodrug, isotope or protected form thereof, wherein R¹, R² and R³ are each
independently selected from: H, methyl and optionally substituted C1-6 alkyl, C1-6 alkenyl or C1-6 alkynyl and wherein the connecting semi-circular line indicates one or more linking atoms/groups comprising a carboxyl group as shown.

6. The composition for use according to claim 1, wherein the cyclic guanidine has the formula III or IIIa: 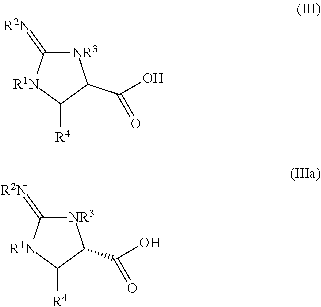
or a pharmaceutically acceptable salt, derivative, solvate, isomer, tautomer, N-oxide, ester, prodrug, isotope or protected form thereof, wherein R¹, R², R³ and R⁴ are each independently selected from: H and methyl.

7. The composition for use according to claim 6, wherein at least one of R¹, R², R³ and R⁴ is methyl, optionally wherein one of R¹, R², R³ and R⁴ is methyl, the others being H.

8. The composition for use according to claim 6, wherein the cyclic guanidine has the formula III′ or IIIa′: 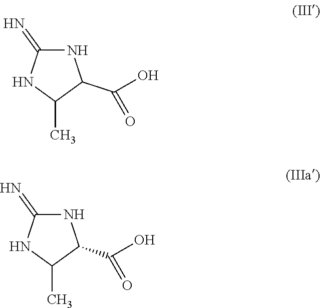
or a pharmaceutically acceptable salt, derivative, solvate, isomer, tautomer, N-oxide, ester, prodrug, isotope or protected form thereof.

9. The composition for use according to claim 1, wherein the cyclic guanidine has the formula IV or IVa: 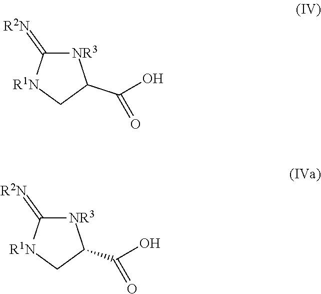
or a pharmaceutically acceptable salt, derivative, solvate, isomer, tautomer, N-oxide, ester, prodrug, isotope or protected form thereof, wherein R¹, R² and R³ are each independently selected from: H and methyl, optionally wherein at least one, or one only, of R¹, R² and R³ is methyl.

10. The composition for use according to claim 1, wherein the cyclic guanidine has the formula V or Va: 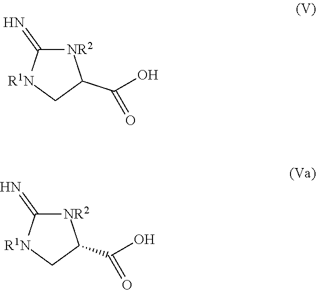
or a pharmaceutically acceptable salt, derivative, solvate, isomer, tautomer, N-oxide, ester, prodrug, isotope or protected form thereof, wherein R¹ and R² are each independently selected from: H and methyl, optionally wherein at least one, or one only, of R¹ and R² is methyl.

11. The composition for use according to claim 1, wherein the cyclic guanidine has the formula VI or VIa: 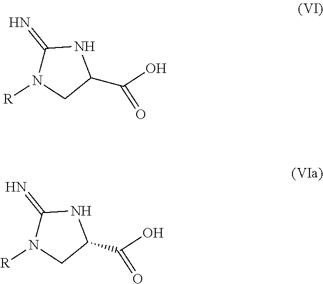
or a pharmaceutically acceptable salt, derivative, solvate, isomer, tautomer, N-oxide, ester, prodrug, isotope or protected form thereof, wherein R is H or methyl.

12. The composition for use according to claim 1, wherein the cyclic guanidine has the formula VII or VIIa: 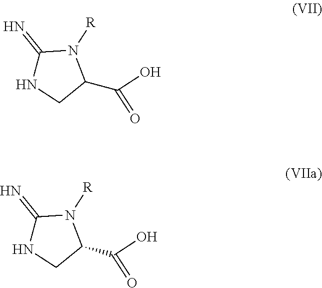
or a pharmaceutically acceptable salt, derivative, solvate, isomer, tautomer, N-oxide, ester, prodrug, isotope or protected form thereof, wherein R is H or methyl.

13-15. (canceled)

16. A composition comprising a cyclic guanidine glucocerebrosidase activator.

17-30. (canceled)

31. A method of treating or preventing a GBA deficiency disease, GCase deficiency, for example in the treatment or prevention of neurodegeneration caused by GCase deficiency, a Lewy body disease (LBD), a Parkinson's disease (PD) or dementia with Lewy bodies (DLB), Alzheimer's disease (AD) or Gaucher's disease (GD), or of inhibiting methanogenic archaea in the digestive tract, comprising administering an effective amount of a composition or a cyclic guanidine as defined in claim 1 to a subject, for example to a human subject.

32. (canceled)

33. A process for producing a yogurt, or for monitoring the quality of a yogurt, comprising the steps of:
(a) providing a yogurt sample; and
(b) testing the sample for GCase activity and/or for the presence of glycosidase modulatory activity, for example for the inhibition or promotion of one or more glycosidases.

34. A process for producing a yogurt, or for monitoring the quality of a yogurt, comprising the steps of:
(a) providing a yogurt sample; and
(b) testing the sample for the presence or absence of a cyclic guanidine and/or to measure the amount of a cyclic guanidine in the sample.

35. A process for producing a yogurt, or for monitoring the quality of a yogurt, comprising the steps of:
(a) providing a yogurt sample;
(b) testing the sample for GCase activity and/or for the presence of glycosidase modulatory activity, for example for the inhibition or promotion of one or more glycosidases; and
(c) testing the sample for the presence or absence of a cyclic guanidine, and/or to measure the amount of a cyclic guanidine in the sample.

36. A process for producing a yogurt, comprising the steps of:
(a) providing a milk product;
(b) fermenting the milk product, for example with bacteria comprising Lactobacillus delbrueckii subsp. bulgaricus, to yield a yogurt;
(c) providing a sample of the yogurt of step (b);
(d) testing the sample:
(i) for GCase activity; and/or
(ii) for the presence or absence of a cyclic guanidine; and/or
(iii) to measure the amount of a cyclic guanidine in the sample; and/or
(iv) for the presence of glycosidase modulatory activity, for example for the inhibition or promotion of one or more glycosidases.

37. A process for producing a yogurt, or for monitoring the quality of a yogurt, comprising the steps of:
(a) providing a yogurt sample;
(b) testing the sample for the presence of glycosidase modulatory activity, for example for the inhibition or promotion of one or more glycosidases.

38-40. (canceled)

41. A process for producing a cyclic guanidine composition as defined in claim 1, comprising the steps of:
(a) providing a yogurt sample; and
(b) extracting the yogurt sample with a polar solvent to yield a fraction enriched in said cyclic guanidine.

42-44. (canceled)
